SEQUENCE LISTING (28 sequences)

SEQ ID NO: 1 - protein, 16 aa, Pestalotia
  MISC_FEATURE (3)..(3): Xaa = L or M
TAXGXRXLRCHGYNVS

SEQ ID NO: 2 - DNA, 34 bp, Pestalotia
  misc_feature: Primer 210900j2
ggsytscgsaysctscgsctscayggntayaayg

SEQ ID NO: 3 - protein, 16 aa, Pestalotia
  MISC_FEATURE (3)..(3): Xaa = L or M
TAXGXRXLRCHGYNVS

SEQ ID NO: 4 - DNA, 36 bp, Pestalotia
  misc_feature (28)..(28): n = a, c, g, or t
gtasccgtgsagscgsagsrtscgsarnccsakngc

SEQ ID NO: 5 - protein, 14 aa, Pestalotia
  MISC_FEATURE (3)..(3): Xaa = K or E
HFXXEIKXALDYVY

SEQ ID NO: 6 - DNA, 42 bp, Pestalotia
  misc_feature (39)..(39): n = a, c, g or t
cacttcragvaggagatcaaggrsgcsctsgaytaygtntay

SEQ ID NO: 7 - protein, 19 aa, Pestalotia
  MISC_FEATURE (3)..(3): Xaa = S or G
ASXIXCYMKDXPXATEEXA

SEQ ID NO: 8 - DNA, 41 bp, Pestalotia
gcstcytcytcsgtsgcgycsgggtkytcyttcatytcyca

SEQ ID NO: 9 - protein, 9 aa, Pestalotia
YYPPFAGRC

SEQ ID NO: 10 - DNA, 23 bp, Pestalotia
taytayccsccsttcgcsggscg

SEQ ID NO: 11 - protein, 5 aa, Pestalotia
DFGWG

SEQ ID NO: 12 - DNA, 23 bp, Pestalotia
ttsccccasccgaagtcsacsag

SEQ ID NO: 13 - protein, 9 aa, Pestalotia
  MISC_FEATURE (1)..(1): Xaa = L or P
XLVXQVTRX

SEQ ID NO: 14 - DNA, 27 bp, Pestalotia
  misc_feature (3)..(3): n = a, c, g or t
ttnctsgtsrtccaggtsacscgstts

SEQ ID NO: 15 - protein, 9 aa, Pestalotia
  MISC_FEATURE (1)..(1): Xaa = L or P
XLVXQVTRX

SEQ ID NO: 16 - DNA, 27 bp, Pestalotia
  misc_feature (25)..(25): n = a, c, g or t
waawcgwgtwacctggaywacwagnaa

SEQ ID NO: 17 - protein, 8 aa, Pestalotia
LPSGYYGN

SEQ ID NO: 18 - DNA, 24 bp, Pestalotia
  misc_feature (21)..(21): n = a, c, g or t
ctsccstcsggstaytayggnaay

SEQ ID NO: 19 - protein, 8 aa, Pestalotia
LPSGYYGN

SEQ ID NO: 20 - DNA, 24 bp, Pestalotia
  misc_feature (4)..(4): n = c, g, g or t
rttnccrtartawccwgawggwag

SEQ ID NO: 21 - DNA, 35 bp, Aspergillus
cctgaattcacgcgcgccaacatgtcttccaagtc

SEQ ID NO: 22 - DNA, 31 bp, Aspergillus
gttctcgagctacttattgcgcaccaacacg

SEQ ID NO: 23 - DNA, 18 bp, Aspergillus
accatggcggcactctgc

SEQ ID NO: 24 - DNA, 18 bp, Aspergillus
gagccgtaggggaagtcc

SEQ ID NO: 25 - DNA, 19 bp, Aspergillus
cttcagactgaacctcgcc

SEQ ID NO: 26 - DNA, 20 bp, Aspergillus
gactcggtccgtacattgcc

SEQ ID NO: 27 - DNA, 38 bp, Aspergillus
cctacggctccgagagaggccttttgatccttgcggag

SEQ ID NO: 28 - DNA, 20 bp, Aspergillus
gagttagtagttggacatcc

(19) United States
(12) Patent Application Publication
(10) Pub. No.: US 2004/0038353 A1
(43) Pub. Date: Feb. 26, 2004

(54) Heterologous expression of taxanes

(76) Inventor: Jesper Vind, Lyngby (DK)

Correspondence Address: NOVOZYMES NORTH AMERICA, INC., 500 FIFTH AVENUE, SUITE 1600, NEW YORK, NY, 10110, US

(21) Appl. No.: 10/416,974

(22) PCT Filed: Nov. 16, 2001
(86) PCT No.: PCT/DK01/00763

(30) Foreign Application Priority Data
Nov. 17, 2000 (DK) 2000 01730

(51) Int. Cl. C12P 17/02; C12N 1/21; C12N 1/18; C12N 15/74; C12N 5/04; C12N 15/82; C 07D 3 5/14

(52) U.S. Cl. 435/123; 435/455; 435/468; 435/252.3; 435/254.2; 435/419; 435/320.1; 549/510

(57) ABSTRACT

The present invention relates to a method of heterologous production of a taxane or a related compound by cloning a DNA sequence comprising a taxane synthesis pathway, making a DNA construct wherein said DNA sequence is under control of regulatory elements, introducing said DNA construct into a host cell, growing said host cell under conditions conductive to the production of the taxane in question, and recovering the taxane in question from the culture medium. The invention also relates to the DNA sequence comprising the taxane synthesis pathway, an expression vector comprising the taxane synthesis pathway, and a host cell comprising the expression vector comprising the taxane synthesis pathway being capable of heterologous expression of taxane.

### FIELD OF THE INVENTION

The present invention relates to a method of heterologous production of a taxane, a DNA sequence comprising the taxane synthesis pathway, an expression vector comprising the taxane synthesis pathway, a host cell comprising the expression vector comprising the taxane synthesis pathway being capable of heterologous expression of taxane, in particular taxol or a related taxane.

### BACKGROUND OF THE INVENTION

The group of complex terpene-type compounds known as taxanes and taxane-related compounds have proven to have important anti-cancer properties. Examples of such compounds include taxol, baccatin and cephalomannine.

Two very commercially important taxanes are the anti-cancer drug taxol and taxotere. The generic name for taxol is PACLITAXEL™, which is now registered as a trade name by Bristol-Myers Squib). The generic name for taxotere is DOCETAXEL™, which is now a registered trade name from Rhone-Poulene Rorer.

Taxanes are very expensive to produce due to a very low production yields obtained from taxane-producing microorganisms, plants or trees.

A number of attempts have been made to synthesis taxol and identify other organisms than Taxus yew tree, which express taxol. Some fungi, such as Pestalotiopsis microspora (Strobel et al. (1996), Microbiology, 142, 435-440); Pestalotia heterocornis (Biotechnology and Bioengineering, Vol. 64, No. 5; and Taxomyces andreanae (Cragg et al. (1993) Nat. Prod. 56, 1657-1688) growing on yew trees has been shown to also express taxol or a related compound. These fungi can be fermented, but the yields are very low.

Little is known about the toxicity of the side-products from these fungi, which may be present in untraceable amounts making homologous expression of taxanes, such as taxol, risky.

The object of the present invention is to provide an alternative to producing taxanes synthetically and homologously from taxane-producing microorganisms.

Wani et al. “Journal of the American Chemical Society” Vol. 93, May 1971, No. 9, pages 2325-2327 reports the structure of taxol and its potential use as an antileukemic and tumor inhibitory compound.

Hezari et al. (1997), Planta Medica, 63, p. 291-295, discloses the single steps in the taxol synthesis pathway.

Strobel et al. (1996), Microbiology, 142, 435-440, disclose that the filamentous fungus Pestalotiopsis microspora isolated from the inner bark of a small limb of Himalayan yew, Taxus wallachiana produce taxol.

U.S. Pat. No. 5,958,741 concerns a method of homologous production of Taxol (and related Taxanes) in fungal microorganisms.

### SUMMARY OF THE INVENTION

The object of the present invention is to provide a method of heterologous expression of taxanes and related compounds, and a host cell capable of producing a taxane, in particular taxol.

The present inventor has provided a method for heterologous expression of taxanes and related compounds. In a preferred embodiment the cloned full-length taxol synthesis pathway from the filamentous fungus Pestalotia heterocornis is transformed, in a vector comprising the taxol synthesis pathway, into a strain of the genus Aspergillus, in particular Aspergillus oryzae host cell, and taxol is expressed heterologously.

Method

In the first aspect the invention relates to a method of heterologous expression of a taxane by cloning a full-length taxane synthesis pathway from a taxane-production organism, plant or tree into a taxane-resistant host cell capable for expressing the taxane in question.

The method of the invention may result in improved yields in comparison to producing the taxane in question homologously and directly from the taxane-producing organisms, plant or tree. It is preferred to use host cells, which do not express any toxins. This way the invention provides a safer taxane production method in comparison to homologous taxane production.

Fungal Host Cell

In the second aspect the invention relates to a taxane-resistant host cell comprising a full-length taxane synthesis pathway wherein the taxane synthesis pathway is foreign to the host cell.

In a preferred embodiment the host cell in question do not produce any toxins.

The host cell is of microbial or plant origin. Particularly contemplated microbial host cells are of fungal or bacterial origin, especially of yeast and filamentous fungi origin, especially the yeast Saccharomyces cerevisiae or the filamentous fungi of the genus Aspergillus, in particular A. oryzae. Specifically contemplated strains include A. oryzae JaL250 and A. oryzae JaL355.

Taxanes and Related Compounds

According to the invention taxane, which may be referred to as a terpene-type compound, in particular a diterpene-type compound, is defined as a chemical compound of the general structure shown below as formulae (I):

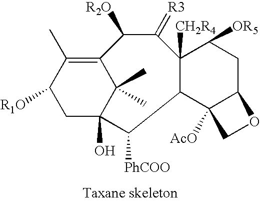

wherein

R1 is a hydrogen atom, an acyl group, or a glycosyl group;

R2 is a hydrogen atom or an acyl group;

R3 is an oxygen atom or the combination of an acetoxyl or hydroxyl group with an hydrogen atom;

R4 is an hydrogen atom or an hydroxyl group;

R5 is a hydrogen atom, an acyl group or a glycosyl group;

Ph is a phenyl group; and

Ac is an acetyl group.

In an embodiment the taxane is selected from the group of 10-deacetylbaccatin III (10-Dab), baccatin III, Cephalomannine or other known taxanes, preferably having pharmaceutical properties.

In an even more preferred embodiment the taxane in question is taxol, which has the chemical structural formulae (II):

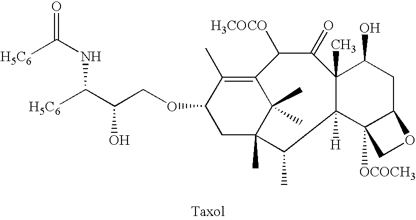

Other taxanes include taxanes known from the taxol synthesis pathway described by Hezari et al. (1997), Planta Medica 63, p. 291-295.

### BRIEF DESCRIPTION OF THE DRAWING

FIG. 1 shows an alignment of a number of sequences with a high degree of homology to the Taxadiene synthase sequence (Id W31655).

FIG. 2 shows an alignment of a number of sequences with high degree of homology to the Taxa-4(20),11(12)-dien-5alpha-ol-0-acetyl transferase sequence (Id q9m6f0).

### DETAILED DESCRIPTION OF THE INVENTION

The present invention relates to a method of heterologous production of a taxane or a related compound.

The term “heterologous” expression or production means that the DNA construct comprising the pathway genes involved in the taxane expression is introduced into a host cell of a species, which is different from the taxane-producing organism (donor cell) from which the taxane pathway originates. In other words, the taxane pathway is foreign to the host cell.

According to a specific embodiment of the invention the full-length taxol synthesis pathway is isolated from the filamentous fungus Pestalotia heterocornis and introduced into a fungal host cell capable of expressing taxol. This can be used to express any taxane or taxane related compound, in particular Taxol, heterologously.

### Methods of Cloning a Taxane Synthesis Pathway

Techniques used to isolate or clone a DNA sequence comprising a taxane synthesis pathway are known in the art and include isolation from genomic DNA, preparation from cDNA, or a combination thereof.

The full-length taxane synthesis pathway (i.e., a full-length taxane gene cluster responsible for taxane expression in a taxane-producing organism, plant, or tree) may for instance be cloned by what is referred to as “Expression Cloning” or by well-known cloning techniques based on conserved regions.

obtaining the Taxane Systhesis Pathway

The taxane synthesis pathway may be obtained from any taxane-producing organism, plant or tree known in the art including the below mentioned.

Examples of microorganisms from which a taxane synthesis pathway may be obtained/cloned include a strain of the genus Pestalotiopsis, in particular a strain of Pestalotiopsis microspora; a strain of the genus Pestalotia, in particular a strain of Pestalotia heterocornis.

Expression Cloning

A number of expression cloning methods are known in the art including WO 99/32617. Another suitable example of such an Expression cloning method is described in WO 93/11249 (from Novo Nordisk), which is hereby incorporated by reference. The method comprises the steps of:

a) cloning, in suitable vectors, a DNA library from an organism suspected of producing one or more proteins of interest;

b) transforming suitable yeast host cells with said vectors;

c) culturing the host cells under suitable conditions to express any protein of interest encoding by a clone in the DNA library; and

d) screening for positive clones by determining any activity of a protein expressed in step c).

Conserved Region Cloning

The cloning of the nucleic acid sequences comprising a taxane synthesis pathway from genomic DNA from a taxane-producing organism, plant or tree, can be effected, e.g., by using the well-known polymerase chain reaction (PCR) or antibody screening of expression libraries to detect cloned DNA fragments with shared structural features. See, e.g., Innis et al., 1990, A Guide to Methods and Application, Academic Press, New York. Other nucleic acid amplification procedures such as ligase chain reaction (LCR), ligated activated transcription (LAT) and nucleic acid sequence-based amplification (NASBA) may be used.

The term “isolated” nucleic acid (DNA) sequence as used herein refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure, as determined by agarose gel electorphoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment(s) comprising the nucleic acid sequence(s) from the taxane synthesis pathway, in particular taxol synthesis pathway, insertion of the fragment into a vector, and incorporation of the recombinant vector into a host cell where multiple copies or clones of the nucleic acid sequence will be replicated. The nucleic acid sequence may be of genomic, cDNA, RNA, semi-synthetic, synthetic origin, or any combinations thereof.

Cloning Based on Known Pathway Genes

Known genes or parts thereof from a taxane synthesis pathway, in particular the taxol synthesis pathway, may be used to design an oligonucleotide probe, which can be used to isolate the full-length taxane synthesis pathway from a taxane-producing organism, plant or tree. Further, such probes can also be used for hybridization with the genomic or cDNA of other taxane-producing organisms, plants or trees, following standard Southern blotting procedures, in order to identify and isolate the corresponding or related taxane synthesis pathways.

A number of genes from the taxol synthesis pathway or a related taxane synthesis pathway suitable as starting point for cloning a full-length taxane synthesis pathway, in particular the taxol synthesis pathway, are known. Walker et al. (2000), Archives of Biochemistry and Biophysics, Vol. 374, No. 2, pp. 371-380 discloses how to clone taxa-4(20),11(12)-dien 5alpha-ol-O-acetyl transferase cDNA from Taxus cells and functional expression in E. coli. Taxa-4(20),11(12)-dien5alpha-ol-O-acetyl transferase is an enzyme which catalyses the third step in the taxol synthesis pathway and is thus a suitable starting point for cloning the full-length pathway of taxol and other relates taxanes.

Further, Williams et al. (2000), Archives of Biochemistry and Biophysics, Vol. 379, No. 1, pp. 137146 discloses heterologous expression of the diterpene cyclase taxadiene synthase from yew (Taxus) species involved in the taxol pathway. The gene encoding this enzyme may also be used for cloning the full-length pathway according to the invention.

Probes for cloning the full-length pathway can be considerably shorter than the entire sequence, but should be at least 15, preferably at least 25, and more preferably at least 40 nucleotides in length. Longer probes can also be used. Both DNA and RNA probes can be used. The probes are typically labeled for detecting the corresponding gene (for example, with ³²p, ³H, ³⁵S, biotin, or avidin). A PCR reaction using the degenerate probes mentioned herein and genomic DNA or first-strand cDNA from, e.g., Pestalotia heterocornis or Pestalotiopsis microspora, can also be used as a probe to clone the corresponding genomic or cDNA.

Introduction of the Taxane Synthesis Pathway Into a Host Cell

When the taxane synthesis pathway nucleic acid (DNA) sequence has been cloned or isolated it is inserted into a suitable nucleic acid construct, especially an expression vector, which is introduced into a host cell using standard techniques know in the art. In the case of Aspergillus oryzae a suitable method is disclosed in EP 238,023-B1.

Nucleic Acid Sequence Encoding the Taxane Synthesis Pathway

The present invention also relates to nucleic acid (DNA) constructs comprising a taxane synthesis pathway nucleic acid sequence, in particular the taxol synthesis pathway, responsible for taxane expression, in particular taxol expression.

The present nucleic acid constructs comprises the taxane synthesis pathway nucleic acid sequence in question, in particular the taxol synthesis pathway. In one embodiment one or more of the genes in the pathway is(are) operably linked to one or more control sequences capable of directing the expression of the coding sequences in a suitable host cell under conditions compatible with the control sequences. “Nucleic acid construct” (or “DNA construct”) is defined herein as a nucleic acid molecule, either single- or double-stranded, which is isolated from naturally occurring gene(s), which has been modified to contain segments of nucleic acids, which are combined and juxtaposed in a manner, which would not otherwise exist in nature. The term nucleic acid construct may be synonymous with the term expression cassette when the nucleic acid construct contains all the control sequences required for expression of coding sequence(s) involved in a taxane synthesis pathway.

The term “coding sequence(s)” as defined herein refer to the sequence(s), which is(are) transcribed into mRNA and translated into the protein/enzyme involved in the taxane synthesis, in particular taxol synthesis, when placed under the control of the above mentioned control sequences.

It is to be understood that the single (individual) genes encoding proteins involved in mediating/catalysing taxane synthesis may be regulated by the same or different control sequences, such as the native control sequence(s) regulating the single (individual) genes in the taxane synthesis pathway in question.

The boundaries of the coding sequence are generally determined by a translation start codon ATG at the 5′-terminus and a translation stop codon at the 3′-terminus. A coding sequence can include, but is not limited to, DNA, cDNA, and recombinant nucleic acid sequences.

An isolated nucleic acid sequence encoding a protein involved in taxane synthesis may be manipulated in a variety of ways to provide for improved taxane expression, in particular taxol expression. Manipulation of the nucleic acid sequence encoding a protein in the taxane synthesis pathway in question prior to its insertion into a vector may be desirable or necessary depending on the expression vector. The techniques for modifying nucleic acid sequences utilizing cloning methods are well known in the art.

### Control Sequences

The term “control sequences” is defined herein to include all components, which are necessary or advantageous for expression of the coding sequence of the nucleic acid sequence of the invention. Each control sequence may be native or foreign to the nucleic acid sequence encoding the protein involved in taxane synthesis. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

### Promoters

The control sequence may be an appropriate promoter sequence, a nucleic acid sequence, which is recognized by a host cell for expression of the nucleic acid sequence. The promoter sequence contains transcription and translation control sequences, which mediate the expression of the protein involved in taxane synthesis. The promoter may be any nucleic acid sequence, which shows transcriptional activity in the host cell of choice and may be obtained from genes encoding extracellular or intracellular polypeptides either homologous or heterologous to the host cell.

Bacterial Promoters

Examples of suitable promoters for directing the transcription of the nucleic acid constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus licheniformis alpha-amylase gene (amyL), the Bacillus stearothermophilus maltogenic amylase gene (amyM), the Bacillus amyloliquefaciens alpha-amylase gene (amyQ), the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences. USA 75:3727-3731), as well as the tac promoter (DeBoer et al., 1983, Proceedings of the National Academy of Sciences USA 80:21-25). Further promoters are described in “Useful proteins from recombinant bacteria” in Scientific American, 1980, 242:74-94; and in J. Sambrook, E. F. Fritsch, and T. Maniatus, 1989, Molecular Cloning, A Laboratory Manual, 2d edition, Cold Spring Harbor, N.Y.).

Fungus Promoters

Examples of suitable promoters for directing the transcription of the nucleic acid constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oxyzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral alpha-amylase and Aspergillus oryzae triose phosphate isomerase), and glaA promoters.

Yeast Promoters

In a yeast host, useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488.

### Transcription Terminators

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator, which is functional in the host cell of choice, may be used in the present invention.

Fungus Terminators

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Yeast Terminators

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, Yeast 8:423-488. Terminator sequences are well known in the art for mammalian host cells.

### Leader Sequences

The control sequence may also be a suitable leader sequence, a non-translated region of mRNA, which is important for translation by the host cell. The leader sequence is operably linked to the 5′ terminus of the nucleic acid sequence encoding the polypeptide in question. Any leader sequence, which is functional in the host cell of choice, may be used according to the present invention.

Fungus Leader Sequences

Preferred leaders for filamentous fungal host cells are obtained from the genes encoding Aspergillus oryzae TAKA amylase and Aspergillus oxyzae triose phosphate isomerase.

Yeast Leader Sequences

Suitable leaders for yeast host cells are obtained from the Saccharomyces cerevisiae enolase (ENO-1) gene, the Saccharomyces cerevisiae 3-phosphoglycerate kinase gene, the Saccharomyces cerevisiae alpha-factor, and the Saccharomyces cerevisiae alcohol dehydrogenase/glyceraldehyde-3-phosphate dehydrogenase genes (ADH2/GAP).

### Polyadenylation Sequences

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA. Any polyadenylation sequence, which is functional in the host cell of choice, may be used according to the present invention.

Fungus Polyadenylation Sequences

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Yeast Polyadenylation Sequences

Useful polyadenylation sequences for yeast host cells are described by Guo and Sherman, 1995, Molecular Cellular Biology 15:5983-5990. Polyadenylation sequences are well known in the art for mammalian host cells.

### Signal Peptide

The control sequence may also be a signal peptide-coding region, which codes for an amino acid sequence linked to the amino terminus of a protein, which can direct the expressed protein into the cell's secretory pathway. The 5′ end of the coding sequence of the nucleic acid sequence may inherently contain a signal peptide-coding region naturally linked in translation reading frame with the segment of the coding region, which encodes the secreted protein. Alternatively, the 5′ end of the coding sequence may contain a signal peptide-coding region, which is foreign to that portion of the coding sequence, which encodes the secreted protein. The foreign signal peptide-coding region may be required where the coding sequence does not normally contain a signal peptide-coding region. Alternatively, the foreign signal peptide-coding region may simply replace the natural signal peptide-coding region in order to obtain enhanced secretion of the protein(s) relative to the natural signal peptide-coding region normally associated with the coding sequence. The signal peptide-coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed protein into the secretory pathway of a host cell of choice may be used according to the present invention.

Bacterial Signal Peptide Sequences

An effective signal peptide-coding region for bacterial host cells is the signal peptide-coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprs, nprM), and the Bacillus subtilis PrsA gene. Further signal peptides are described by Simonen and Palva, 1993, Microbiological Reviews 57:109-137.

Fungus Signal Peptide Sequences

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase gene, or the Rhizomucor miehei lipase gene.

Yeast Signal Peptide Sequences

Useful signal peptides for yeast host cells are obtained from the genes for Saccharomyces cerevisiae alpha-factor and Saccharomyces cerevisiae invertase. Other useful signal peptide coding regions are described by Romanos et al., 1992, Yeast 8:423-488.

### Propeptide Sequences

The control sequence may also be a propeptide coding region, which codes for an amino acid sequence positioned at the amino terminus of a protein. The resultant protein is known as a proenzyme or propolypeptide (or a zymogen in some cases). A propolypeptide is generally inactive and can be converted to mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

The nucleic acid constructs of the present invention may also comprise one or more nucleic acid sequences, which encode one or more factors that are advantageous in the expression of the polypeptide, e.g., an activator (e.g., a trans-acting factor), a chaperone, and a processing protease. Any factor that is functional in the host cell of choice may be used according to the present invention. The nucleic acids encoding one or more of these factors are not necessarily in tandem with the nucleic acid sequence encoding the polypeptide.

An activator is a protein, which activates transcription of a nucleic acid sequence encoding a polypeptide (Kudla et al., 1990, EMBO Journal 9:1355-1364; Jarai and Buxton, 1994, Current Genetics 26:2238-244; Verdier, 1990, Yeast 6:271-297). The nucleic acid sequence encoding an activator may be obtained from the genes encoding Bacillus stearothexmophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra, and MacKenzie et al., 1993, Journal of General Microbiology 139:2295-2307.

A chaperone is a protein, which assists another polypeptide in folding properly (Hartl et al., 1994, TIBS 19:20-25; Bergeron et al., 1994, TIBS 19:124-128; Demolder et al., 1994, Journal of Biotechnology 32:179-189; Craig, 1993, Science 260:1902-1903; Gething and Sambrook, 1992, Nature 355:33-45; Puig and Gilbert, 1994, Journal of Biological Chemistry 269:7764-7771; Wang and Tsou, 1993, The FASEB Journal 7:1515-11157; Robinson et al., 1994, Bio/Technology 1:381-384). The nucleic acid sequence encoding a chaperone may be obtained from the genes encoding Bacillus subtilis GroE proteins, Aspergillus ozyzae protein disulphide isomerase, Saccharomyces cerevisiae calnexin, Saccharomyces cerevisiae BiP/GRP78, and Saccharomnyces cerevisiae Hsp70. For further examples, see Gething and Sambrook et al, 1989, Molecular Cloning, A Laboratory Manual, 2d edition, Cold Spring Harbor, N.Y., and Hartl et al., 1994, TIBS 19:20-25.

A processing protease is a protease that cleaves a propeptide to generate a mature biochemically active polypeptide (Enderlin and ogrydziak, 1994, Yeast 10:67-79; Fuller et al., 1989, Proceedings of the National Academy of Sciences USA 86:1434-1438; Julius et al., 1984, Cell 37:1075-1089; Julius et al., 1983, Cell 32:839-852). The nucleic acid sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xprx6).

### Regulatory Sequences

It may also be desirable to add regulatory sequences, which allow the regulation of the expression of one or more polypeptides involved in the pathway relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GAL1 system may be used.

In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those, which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding a polypeptide involved in the taxane pathway would be placed in tandem with the regulatory sequence.

### Expression Vectors

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence(s) is(are) located in the vector so that the coding sequence is operably linked with the appropriate control sequences for expression, and possibly secretion.

The recombinant expression vector may be any vector (e.g., a plasmid or virus), which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, a cosmid or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, chloramphenicol or tetracycline resistance. A frequently used mammalian marker is the dihydrofolate reductase gene. Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Preferred for use in an Aspergillus cell are the amds and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

A vector of the present invention preferably contain an element(s) that permits stable integration of the vector into the host cell genome or autonomous replication of the vector in the cell independent of the genome of the cell.

A vector of the present invention may be integrated into the host cell genome when introduced into a host cell. For integration, the vector may rely on the nucleic acid sequence encoding (a) polypeptide(s) involved in the taxane synthesis pathway or any other element of the vector for stable integration of the vector into the genome by homologous or none homologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient-number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination. These nucleic acid sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUCl9, pACYCI77, pACYC184, pUB110, pEl94, pTA1060, and pAMB1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

The episomal replicating AMA1 plasmid vector disclosed in WO 00/24883 may also be used.

More than one copy of a nucleic acid sequence encoding polypeptide(s) involved in the taxane synthesis pathway of the present invention may be inserted into the host cell to amplify expression of the nucleic acid sequence. Stable amplification of the nucleic acid sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al, 1989, Molecular Cloning, A Laboratory Manual, 2d edition, Cold Spring Harbor, N.Y.).

### Host Cells

The present invention also relates to recombinant host cells, comprising a nucleic acid sequence of the invention, which are advantageously used in the heterologous (recombinant) production of taxanes and taxane related compounds, preferably taxol: The term “host cell” encompasses any progeny of a parent cell, which is not identical to the parent cell due to mutations that occur during replication. In one embodiment the host cell is taxane-resistant. Taxane-resistance can be engineered into the host by a functional taxane-resistant beta-tubulin encoding gene into the host. The functional taxane-resistant beta-tubulin encoding gene could preferably be a variant of the host beta-tublin encoding gene, preferably mutated in position Leu-215, Leu-217 and/or Leu-228, as found in beta-tubulin in Chinese hamster ovary cells (Gonzalez-garay M. L., Chang L., Blade K., Menick D. R, Cabral F. (1999) vol 274 pp23875-23882). These are preferably mutated to His, Arg or Phe.

The cell is preferably transformed with a vector comprising a nucleic acid sequence of the invention followed by integration of the vector into the host chromosome. “Transformation” means introducing a vector comprising a nucleic acid sequence of the present invention into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination as described above.

Prokaryote Host cells

The microbial host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell.

Transformation of Prokaryote Host Cells

The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

Eukaryote Host Cells

The host cell may be a eukaryote, such as a mammalian cell, an insect cell, a plant cell or a fungal cell. Useful mammalian cells include Chinese hamster ovary (CHO) cells, HeLa cells, baby hamster kidney (BHK) cells, COS cells, or any number of other immortalized cell lines available, e.g., from the American Type Culture Collection.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

In a more preferred embodiment, the yeast host cell is a cell of a species of Candida, Kluyveromyces, Saccharomyces, Schizosaccharomyces, Pichia, or Yarrowia. In a most preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred embodiment, the yeast host cell is a Kluyveromyces lactis cell. In another most preferred embodiment, the yeast host cell is a Yarrowia lipolytica cell.

In a preferred embodiment, the fungal host cell is a filamentous fungal cell. “Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK. The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioldes, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another prefered erbodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell. In another most preferred embodiment, the Trichoderma cell is a Trichoderma harzianum, Trichoderma koningii, Trichoderma longibrachiatum, Trichoderma reesei or Trichoderma viride cell.

Transformation of Eukaryote Host cells

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81:1470-1474. A suitable method of transforming Fusarium species is described by Malardier et al., 1989, Gene 78:147-156 or in copending U.S. Ser. No. 08/269,449. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920. Mammalian cells may be transformed by direct uptake using the calcium phosphate precipitation method of Graham and Van der Eb (1978, Virology 52:546).

Cultivation of Host Cells

The methods used for cultivation of microbial or plant host cells are known in the art.

Plants

The present invention also relates to a transgenic plant, plant part, or plant cell, which has been transformed with a taxane synthesis pathway so as to express and produce taxanes or taxane related compounds, in particular taxol, in recoverable quantities. The taxane in question may be recovered from the plant or plant part. Alternatively, the plant or plant part containing the recombinant taxane in question may be used directly a therapeutic compound.

The transgenic plant can be dicotyledonous (a dicot) or monocotyledonous (a monocot). Examples of monocot plants are grasses, such as meadow grass (blue grass, Poa), forage grass such as festuca, lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rapeseed, and the closely related model organism Arabidopsis thaliana.

Examples of plant parts are stem, callus, leaves, root, fruits, seeds, and tubers. Also specific plant tissues, such as chloroplast, apoplast, mitochondria, vacuole, peroxisomes, and cytoplasm are considered to be a plant part. Furthermore, any plant cell, whatever the tissue origin, is considered to be a plant part.

Also included within the scope of the present invention are the progeny of such plants, plant parts and plant cells.

The transgenic plant or plant cell expressing a taxene or taxane related compound may be constructed in accordance with methods known in the art. Briefly, the plant or plant cell is constructed by incorporating one or more expression constructs comprising a taxnane synthesis pathway into the plant host genome and propagating the resulting modified plant or plant cell into a transgenic plant or plant cell.

Conveniently, the expression construct is a nucleic acid construct, which comprises a nucleic acid sequence encoding proteins involved in taxane synthesis operably linked with appropriate regulatory sequences required for expression of the nucleic acid sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences is determined, for example, on the basis of when, where, and how the polypeptide is desired to be expressed. For instance, the expression of the taxane in question may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV promoter may be used (Franck et al., 1980, Cell 21: 285-294). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889, a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941, the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93, or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588.

A promoter enhancer element may also be used to achieve higher expression of the taxane in question in the plant. For instance, the promoter enhancer element may be an intron, which is placed between the promoter and the nucleotide sequence encoding a polypeptide of the present invention. For instance, Xu et al., 1993, Plant Molecular Biology 22: 573-588, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38). However it can also be used for transforming monocots, although other transformation methods are generally preferred for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated therein the expression construct are selected and regenerated into whole plants according to methods well known in the art.

The present invention also relates to methods for producing taxanes or taxane related compounds comprising (a) cultivating a transgenic plant or a plant cell comprising a nucleic acid sequence comprising a taxane synthesis pathway of the present invention under conditions conducive for production of the taxane in question; and (b) recovering the taxane in question.

### Materials & Methods

pYAC4 has been deposited as ATCC67379 at American Type Culture Collection.

Pestalotia heterocornis strain is described by Noh et al. (1999), Biotechnol. Bioeng. Vol 64 pp. 620-623, and has been deposited as no. KCTCO340 Bp at the Korean Collection For Type Cultures)

QIAprep® Spin Miniprep kits, cat no. 27104(QIAGEN, Venlo, The Netherlands)

Aspergillus oryzae Jal250 is a derivative of Aspergillus oryzae A1560 in which the pyrG gene has been inactivated, as described in WO 98/01470

BECh2 is described in WO 00/39322 which is further refer to patent WO 98/12300 (describes JaL228) pJaL173 is described in patent WO 98/12300 pJaL335 is described in patent WO 98/12300

Yeast AB 1380 is deposited as ATCC204682 (American type culture collection

Assay for Determination of Taxol Activity

Monoclonal Antibody-Based Immunoassay system for the quantitative Detection of taxol in biological matrices (from Hawaii Biotechnology Group, Inc.) Cat no. TA02

Assay for Determination of Taxane Activity

Monoclonal Antibody-Based Immunoassay system for the quantitative Detection of taxane in biological matrices (from Hawaii Biotechnology Group, Inc.) Cat no. TA04

### EXAMPLES

### Example 1

### Expression Cloning of the Taxane Synthesis Pathway in pYAC.

Cloning Based on Heterologous Expression:

DNA from Pestalotia heterocornis is prepared using a QIAprep® Miniprep Kit (QIAGEN, Venlo, The Netherlands) in which the procedure provided by the manufacturer is modified. Briefly, the strain is grown in 5 ml YPD for three days. The mycelia is collected by filtration and washed with 200 ml of water, then transferred to a 2 ml microfuge tube and lyophilized by centrifugation under vacuum for three hours at 60° C. The dried mycelia is then ground and re-suspended in one ml of lysis buffer (100 mM EDTA, 10 mM Tris pH 8.0, 1% tritonX-100, 500 mM guanidine-HCl, 200 mM NaCl), followed by thorough mixing. Twenty micro g RNAse is added to each tube, which is then incubated at 37° C. for 10 min. One hundred micro g proteinase K is added, and the reaction is incubated for 30 minutes at 50° C. Each tube is then centrifuged for 15 minutes at top speed in a standard bench top microfuge. The supernatant is applied onto a QIAprep® spin column, then centrifuged and filtrate discarded. The column is then washed in 0.5 ml PB provided in the kit, and centrifuged again for one minute. After the filtrate is discarded, the column is washed in 0.75 ml PE provided in the kit, then centrifuged once more for one minute. The column is allowed to air dry, and the DNA is eluted by addition of 100 micro 1 TE buffer followed by a final one min spin.

The plasmid pYAC4 (obtained from ATCC (American type culture collection) is lineriazed with BamHI and the two restrictions sites are dephosphorylised with alkaline phosphatase (Calf intestinal phosphatase from New England biolabs). The vector is phenol extracted and the vector is cut with EcoRI.

The DNA from Pestalotia heterocornis is partially digested with EcoRI in agarose plugs as described by Albertsen H. M., Paslier D. L. Abderrahim H., Dausset J., Cann H., Cohen D. (1989) Nuc. Acid res. Vol 17 no. 2 pp. 808, by limiting the Mg concentration. The DNA is separated on a CHEF apparatus in a 1% Seaplaque low melting agarose (Albertsen H. M., Abderrehim H., Cann H. M., Dausset J., Paslier D. L., Cohen D., (1990) Proc. Natl. Acad. Sci. USA Vol. 87 pp. 4256-4260).

The agarose plug containing the digested DNA is equilibrated in ligation buffer for 1 hour. The vector is added and the agarose is briefly melted at 68° C. When the agarose has cooled down to 37° C., 10 micro 1 of T4 DNA ligase (400.000 units/ml) is added along with fresh ligation buffer followed by overnight incubation at room temperature. The DNA containing agarose is heated to 68% and treated with agarase for 2 hours at 37° C.

The ligation is immediately transformed into yeast (Strain AB1380) using the spheroplast method Burgess P. M. J., Percival J., (1987), Anal. Biochem. Vol. 163 pp.391-397. The transformations are plated on minimal media (SC-Ura) (M.Ramsey (1994), Molecular Biotechnology V.1 p.181-201).

The yeast transformants are inoculated in growth media (YPD or SC-Ura) and grown for 2-4 days. Production of taxol is detected using the Taxol screening kit as described by the manufacture (developed by Hawaii Biotechnolgy Group Inc. (Cat No. TA02)).

The pYAC4 plasmid are isolated from the yeast transformants using the Nucleobond plasmid kit (from Clontech) and transformed into Aspergillus oryzae, using the general transformation method described below.

The Aspergillus transformants are inoculated in growth media (YPD or S7 (Noh m., Yang J., Kim K., Yoon X., Kang K., Han H., Shim S., Park H. (1999) Biotechnol. Bioeng. vol 64 pp. 620-623) and grown for 4-7 days. Production of taxol is detected using the Taxol screening kit as described by the manufacture (developed by Hawaii Biotechnolgy Group Inc. (Cat No. TA02)).

Transformation of Aspergillus oryzae (General Procedure)

100 ml of YPD (Sherman et al., Methods in Yeast Genetics, Cold Spring Harbor Laboratory, 1981) is inoculated with spores of A. oryzae and incubated with shaking for about 24 hours. The mycelium is harvested by filtration through miracloth and washed with 200 ml of 0.6 M MgSO₄. The mycelium is suspended in 15 ml of 1.2 M MgSO₄, 10 MM NaH₂PO₄, pH=5.8. The suspension is cooled on ice and 1 ml of buffer containing 120 mg of Novozym™ 234, batch 1687, is added. After 5 min., 1 ml of 12 mg/ml BSA (Sigma type H25) is added and incubation with gentle agitation continued for 1.57-2.5 hours at 37° C. until a large number of protoplasts is visible in a sample inspected under the microscope.

The suspension is filtered through miracloth, the filtrate transferred to a sterile tube and overlayed with 5 ml of 0.6 M sorbitol, 100 mM Tris-HC1, pH=7.0. Centrifugation is performed for 15 minutes at 1000 g and the protoplasts are collected from the top of the MgSO₄ cushion. 2 volumes of STC (1.2 M sorbitol, 10 mM Tris-HCl, pH=7.5, 10 mM CaCl₂) are added to the protoplast suspension and the mixture is centrifugated for 5 minutes at 1000 g. The protoplast pellet is resuspended in 3 ml of STC and repelleted. This is repeated. Finally, the protoplasts are resuspended in 0.2-1 ml of STC. 100 micro 1 of protoplast suspension is mixed with 5-25 micro g of p3SR2 (an A. nidulans amdS gene carrying plasmid described in Hynes et al., Mol. and Cel. Biol., Vol. 3, No. 8, 1430-1439, Aug. 1983) in 10 micro 1 of STC. The mixture is left at room temperature for 25 minutes 0.2 ml of 60% PEG 4000 (BDH 29576), 10 mM CaCl₂ and 10 mM Tris-HCl, pH=7.5 is added and carefully mixed (twice) and finally 0.85 ml of the same solution is added and carefully mixed. The mixture is left at room temperature for 25 min., spun at 2.500 g for 15 minutes and the pellet is resuspended in 2 ml of 1.2 M sorbitol. After one more sedimentation the protoplasts are spread on minimal plates (Cove, Biochem. Biophys. Acta 113, (1966), 51-56) containing 1.0 M sucrose, pH=7.0, 10 mM acetamide as nitrogen source and 20 mM CsCl to inhibit background growth. After incubation for 4-7 days at 37° C. spores are picked, suspended in sterile water and spread for single colonies. This procedure is repeated and spores of a single colony after the second reisolation are stored as a defined transformant.

### Example 2

### Expression Cloning of the Taxane Synthesis Pathway in Cosmid

Cloning Based on Heterologous Expression:

Pestalotia heterocornis genomic DNA is fragmented to the size of 30-50 Kb by either partial digestion or digestion with rare cutting enzymes and ligated into a cosmid vector, cut with appropriate restrictions enzyme to ensure compatible DNA ends. This is done as described by Tang L., Shah S., Chung L., Carney J., Katz L., Khosla C., Julien B. (2000) Science vol 287 p.640-642

DNA from Pestalotia heterocornis is prepared using a QIAprep® Miniprep Kit (QIAGEN, Venlo, The Netherlands) in which the procedure provided by the manufacturer is modified. Briefly, the strain is grown in 5 ml YPD for three days. The mycelia is collected by filtration and washed with 200 ml of water, then transferred to a 2 ml microfuge tube and lyophilized by centrifugation under vacuum for three hours at 60° C. The dried mycelia is then ground and re-suspended in one ml of lysis buffer (100, mM EDTA, 10 mM Tris pH 8.0, 1% tritonX-100, 500 mM guanidine-HCl, 200 mM NaCl), followed by thorough mixing. Twenty micro g RNAse is added to each tube, which is then incubated at 37° C. for 10 min. One hundred micro g proteinase K is added, and the reaction is incubated for 30 minutes at 50° C. Each tube is then centrifuged for 15 minutes at top speed in a standard bench top microfuge. The supernatant is applied onto a QIAprep® spin column, then centrifuged and filtrate discarded. The column is then washed in 0.5 ml PB provided in the kit, and centrifuged again for one minute. After the filtrate is discarded, the column is washed in 0.75 ml PE provided in the kit, then centrifuged once more for one minute. The column is allowed to air dry, and the DNA is eluted by addition of 100 micro 1 TE buffer followed by a final one min spin.

The Cosmid Supercos1 (obtained from Stratagene) is lineriazed with BamHI.

The DNA from Pestalotia heterocornis is partially digested with BamHI and cloned into supercosl as described in Wahl et al (1987), Proc. Natl. Acad. Sci. USA vol 84 pp. 2160-2164)

The ligation is immediately transformed into E.coli (e.g., DH10B) by electrotransformation and plated onto ampicillin containing plates.

The cosmids are isolated from the E.coli and transformed into Aspergillus oryzae.

The Aspergillus transformants are inoculated in growth media (YPD or S7 (Noh et al. (1999)) and grown for 4-7 days. Production of taxol is detected using the Taxol screening kit as described by the manufacture (developed by Hawaii Biotechnolgy Group Inc. (Cat No. TA02)).

### Example 3

### Cloning of Taxane Synthesis Pathway Based on Homology and Inverted Long-range PCR Cloning

Cloning of the Taxadiene Synthase

Based on the Taxadiene synthase sequence (Id W31655) a homology search is made using BlastP in the following databases: Swissprot, Trembl, GeneseqP, Fastaler_P, and the following sequences are identified; y06566, w85703, q38710, w85710.

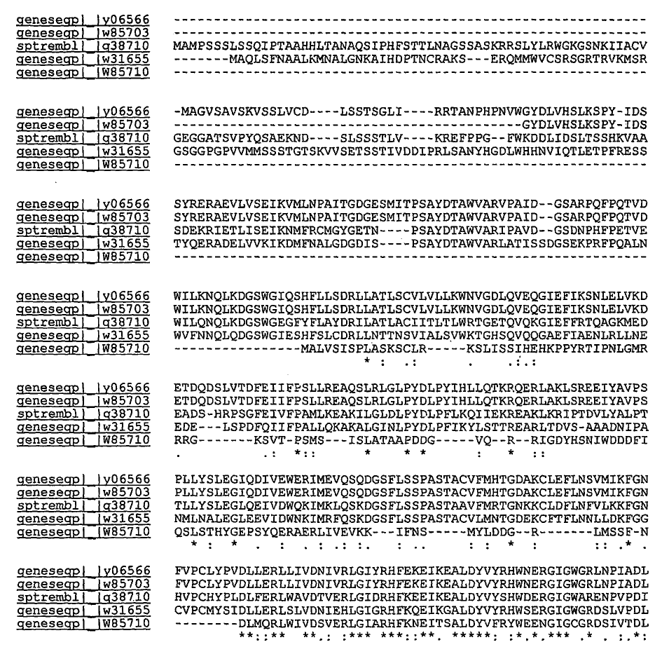

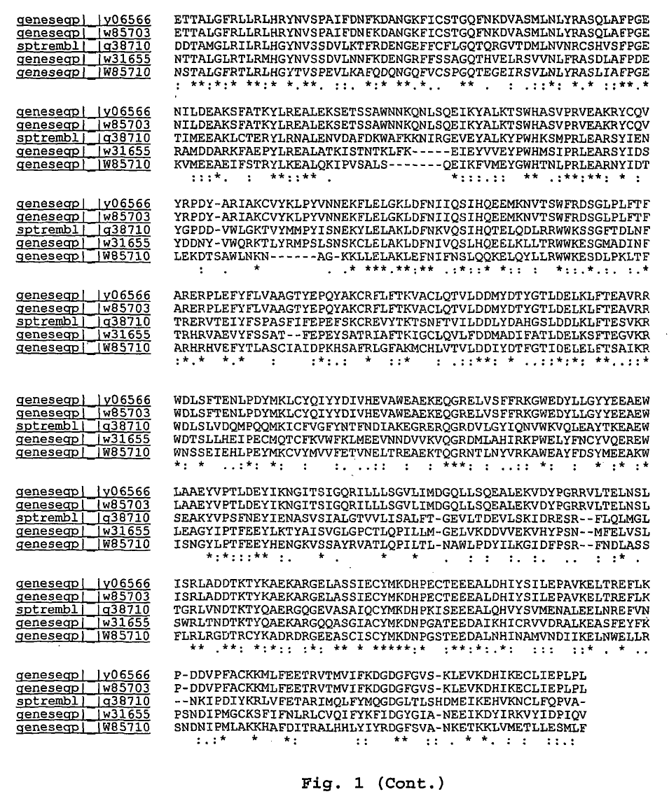

These sequences are aligned using ClustalW. The alignment is shown in FIG. 1.

Based on the conserved region 4 different primers are designed:

Primer210900j2:

(SEQ ID NO: 1)

| | |
|---|---|
| TA(L/M)G(F/L)R(T/I)LRCHGYNVS | (forward) |

(SEQ ID NO: 2)

GGSYTSCGSAYSCTSCGSCTSCAYGGNTAYAAYG

Primer210900j3:

(SEQ ID NO: 3)

| | |
|---|---|
| TA(L/M)G(F/L)R(T/I)LRCHGYNVS | (reverse) |

(SEQ ID NO: 4)

GTASCCGTGSAGSCGSAGSRTSCGSARNCCSAKNGC

Primer210900j1:

| | | |
|---|---|---|
| HF(K/E)(Q/K/E)EIK(G/E)ALDYVY | (forward) | (SEQ ID NO: 5) |
| CACTTCRAGVAGGAGATCAAGGRSGCSCTSGAYTAYGTNTAY | | (SEQ ID NO: 6) |

Primer210900j4:

| | | |
|---|---|---|
| AS(S/G)I(A/E)CYMKD(N/H)P(G/E)ATEE(P/E)A | (reverse) | (SEQ ID NO: 7) |
| GCSTCYTCYTCSGTSGCGYCSGGGTKYTCYTTCATYTCYCA. | | (SEQ ID NO: 8) |

PCR is run using the genomic DNA from Pestalotia heterocornis as template, PWO DNA-polymerase (from Boehringer Mannheim) with the oligoes in following combinations: primer 210900j2 and 210900j4, primer 210900j3 and 210900j1, primer 210900j3 and 210900j4.

The PCR-reactions are run on either a 2% agarose gel (primer 210900j3 and 210900j1) or a 1.5% agarose gel (primer 210900j2 and 210900j4, primer 210900j3 and 210900j4)

Specific bands are isolated from the gel using Qiagen spin columns (Qiaquick gel extraction kit) and cloned using TOPO-cloning kit from Invitrogen.

DNA prep is made from each E. coli transformant and sequenced using the primers in the TOPO-cloning kit.

The Sequences (DNA and the translated amino acid) are aligned to the W31655, to verify the identity.

Based on the DNA sequences at least 2 additional primers are designed and used for Long-range inverted PCR (LR-IPCR).

The LR-IPCR is run using the genomic DNA from Pestalotia heterocornis as template along with designed primers (based on the cloned sequences) essentially as described by Benkel and Fong (1996) Genetic analysis: Biomolecular Engineering vol 13 pp.123-127.

The specific PCR bands are purified from 1% agarose gel and cloned into pTOPO using the TOPO-cloning kit.

The cloned DNA fragments are sequenced using the primer within the TOPO-cloning kit (and primers based on the sequence derived there from (primer walking)).

Based on the new derived sequence additional rounds of LR-IPCR are run and additional flanking DNA sequences are cloned and sequenced (as above).

Cloning of the Taxa-4(20),11(12)-dien-5alpha-ol-0-acetyl Transferase

Based on the Taxa-4(20),11(12)-dien-5alpha-ol-0-acetyl transferase sequence (Id q9m6f0) a homology search is made using BlastP and a number of sequences identified.

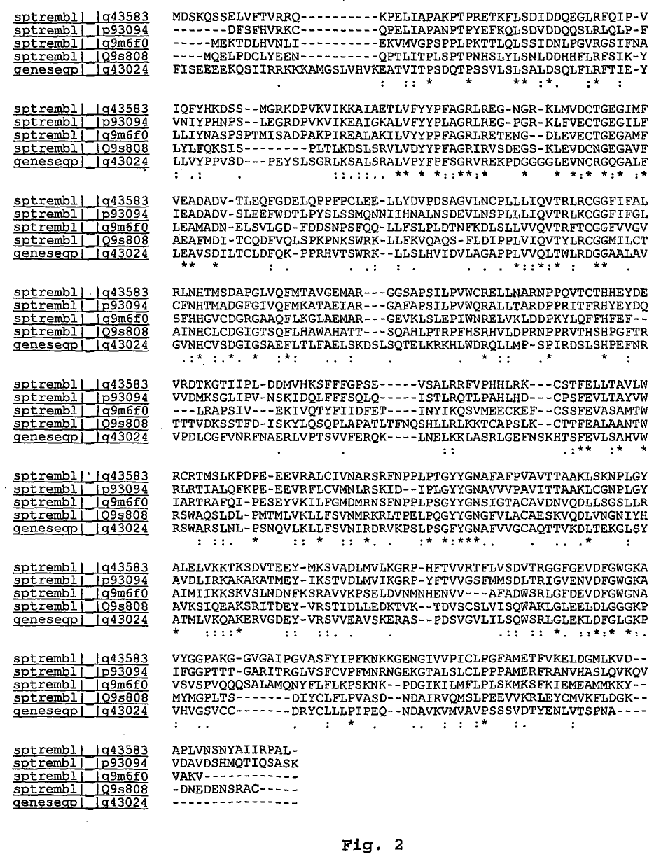

These sequences are aligned using ClustalW. The alignment is shown in FIG. 2.

Based on the conserved region 6 different primers are designed:

| | | | |
|---|---|---|---|
| Primer141100J1: | YYPPFAGRC | (forward) | (SEQ ID NO: 9) |
| | TAYTAYCCSCCSTTCGCSGGSCG | | (SEQ ID NO: 10) |
| Primer200900J4: | DFGWG | (reverse) | (SEQ ID NO: 11) |
| | TTSCCCCASCCGAAGTCSACSAG | | (SEQ ID NO: 12) |
| Primer1411OOJ2: | (L/P)LV(V/I)QVTR(F/L) | (forward) | (SQ ID NO: 13) |
| | TTNCTSGTSRTCCAGGTSACSCGSTTS | | (SEQ ID NO: 14) |
| Primer141100J3: | (L/P)LV(V/I)QVTR(F/L) | (reverse) | (SEQ ID NO: 15) |
| | WAAWCGWGTWACCTGGAYWACWAGNAA | | (SEQ ID NO: 16) |
| Primer141100J4: | LPSGYYGN | (forward) | (SEQ ID NO: 17) |
| | CTSCCSTCSGGSTAYTAYGGNAAY | | (SEQ ID NO: 18) |
| Primer141100J5: | LPSGYYGN | (reverse) | (SEQ ID NO: 19) |
| | RTTNCCRTARTAWCCWGAWGGWAG | | (SEQ ID NO: 20) |

PCR is run using the genomic DNA from Pestalotia heterocornis as template, PWO DNA-polymerase (from Boehringer Mannheim) with the oligoes in following combinations: primer 141100J1 and 200900J4, primer 141100J1 and 141100J3, primer 141100J1 and 141100J5, primer 141100J2 and 200900J4, primer 14110J2 and 141100J5 primer 141100J4 and 200900J4.

The PCR-reactions are run on a 1.5% agarose gel.

Specific bands are isolated from the gel using Qiagen spin columns (Qiaquick gel-extraction kit) and cloned using TOPO-cloning kit from Invitrogen.

DNA prep is made from each E.coli transformant and sequenced using the primers provided in the TOPO-cloning kit.

The Sequences (DNA and the translated amino acid) are aligned to the q9m6f0, to verify the identity.

Based on the DNA sequences at least 2 additional primers are designed and used for Long-range inverted PCR (LR-IPCR).

The LR-IPCR is run using the genomic DNA from Pestalotia heterocornis as template along with designed primers (based on the cloned sequences) essentially as described by Benkel and Fong (1996) Genetic analysis: Biomolecular Engineering vol 13 pp.123-127.

The specific PCR bands are purified from 1% agarose gel and cloned into pTOPO using the TOPO-cloning kit. The cloned DNA fragments are sequenced using the primer within the TOPO-cloning kit (and primers based on the sequence derived there from (primer walking)).

Based on the new derived sequence additional rounds of LR-IPCR are run and additional flanking DNA sequences are cloned and sequenced (as above).

Based on the sequences identified by the Taxadiene synthase cloning and the Taxa-4(20),11(12)-dien-5alpha-ol-0-acetyl transferase cloning, a DNA-fragment containing the gene cluster encoding enzymes involved in Taxane synthesis is cloned into Supercosl and into pYAC4. The DNA-constructs are transformed into Aspergillus.

The Aspergillus transformants are inoculated in growth media (YPD or S7 (Noh et al. (1999)) and grown for 4-7 days. Production of taxol is detected using the Taxol screening kit as described by the manufacture (developed by Hawaii Biotechnolgy Group Inc. (Cat No. TA02)).

### Example 4

Construction of Aspergillus oryzae JaL355

For removing the defect pyrG gene resident in the alkaline protease gene in the A. oryzae strain BECh2 the following was done:

Isolation of a pyrG⁻ A. oryzae strain, ToCl418

The A. oxyzae strain BECh2 was screened for resistance to 5-flouro-orotic acid (FOA) to identify spontaneous pyrG mutants. One strain, ToCI418, was identifying as being pyrG⁻. ToC1418 is uridine dependent, therefore it can be transformed with the wild type pyrG gene and transformants selected by the ability to grow in the absence of uridine.

Construction of a pyrG plus A. oryzae strain, JaL352.

The mutation in the defect pyrg gene resident in the alkaline protease gene was determined by sequencing. Chromosomal DNA from A. oryzae strain BECh2 was prepared and by PCR with primers #104025 (5′-CCTGAATTCACGCGCGCCAACATGTCTTCCAAGTC) (SEQ ID NO: 21) and #104026 (5′-gttctcgagctacttattgcgcaccaacacg) (SEQ ID NO: 22) a 933 bp fragment was amplified containing the coding region of the defect pyrG gene. The 933 bp fragment was purified and sequenced with the following primers: #104025, #104026, #104027 (5′-ACCATGGCGGCACTCTGC) (SEQ ID NO: 23), #104028 (5′-gagccgtaggggaagtcc) (SEQ ID NO: 24), #108089 (5′-CTTCAGACTGAACCTCGCC) (SEQ ID NO: 25), and #108091 (5′-GACTCGGTCCGTACATTGCC) (SEQ ID NO: 26). Sequencing shows that an extra base, a G, was inserted at position 514 in the pyrG-coding region (counting from the A in the start codon of the pyrG gene), thereby creating a frame-shift mutation.

To make an wild type pyrG gene out of the defect pyrG gene resident in the alkaline protease the A. oryzae pyrG⁻ strain ToC1418 was transformed with 150 pmol of the oligo-nucleotide 5′-P-CCTACGGCTCCGAGAGAGGCCTTTTGATCCTTGCGGAG-3′ (SEQ ID NO: 27) using standard produres. The oligo-nucleotide restores the pyrG reading frame, but at the same time a silence mutation is introduce thereby creating a StuI restriction endonuclease site. Transformants were then selected by their ability to grow in the absence of uridine. After reisolation chromosomal DNA was prepared from 8 transformants. To confirm the changes a 785 bp fragment was amplified by PCR with the primers #135944 (5′-GAGTTAGTAGTTGGACATCC) (SEQ ID NO: 28) and #108089, which is covering the region of interest. The 785 bp fragment was purified and sequenced with the primers #108089 and #135944. One strain having the expected changes was named JaL352.

Isolation of a pyrG⁻A. oryzae strain, JaL355

For removing the pyrG gene resident in the alkaline protease gene JaL352 was transformed by standard procedure with the 5.6 kb BamHI fragment of pJaL173 harbouring the 5′and 3′ flanking sequence the A. oryzae alkaline protease gene. Protoplasts were regenerated on non-selective plates and spores were collected. About 10⁹ spores were screened for resistance to FOA to identify pyrG mutants. After reisolation chromosomal DNA was prepared from 14 FOA resistance transformants. The chromosomal DNA was digested with Bal I and analysed by Southern blotting, using the 1 kb ³²P-labelled DNA Bal I fragment from pJaL173 containing part of the 5′ and 3′ flanks of the A. oryzae alkaline protease gene as the probe. Strains of interest were identified by the disappearance of a 4.8 kb Bal I band and the appearance of a 1 kb Bal I band. Probing the same filter with the 3.5 kb ³²P-labelled DNA Hind III fragment from pJaL335 containing the A. oryzae pyrG gene gives that the 4.8 kb Bal I band is disappeared in the strains of interest. One strain resulting from these transformants was named JaL355.

1. A method of heterologous expression of a taxane or a taxane related compound comprising
i) cloning a DNA sequence comprising a taxane synthesis pathway,
ii) making a DNA construct wherein said DNA sequence obtained in step i) is under control of regulatory elements,
iii) introducing said DNA construct into a host cell,
iv) growing said host cell under conditions conductive to the production of the taxane in question, and
v) recovering the taxane in question from the culture medium.

2. The method of claim 1, wherein the method optionally may comprise the step vi) of purifying the recovered taxane product obtained in step v).

3. The method of claims 1 or 2, wherein the host cell is of microbial, in particular fungal or bacterial origin, especially of yeast or filamentous fungus origin, or plant origin

4. The method of claims 1-3, wherein a full-length taxane synthesis pathway, in particular full-length taxol synthesis pathway, is cloned in step i).

5. The method according to claims 1-4, wherein the DNA construct is introduced into a host cell of a species, which is different from the taxane-producing microorganism (donor cell).

6. The method of claims 1-5, wherein the taxane or taxane related compound is taxol

7. The method of claims 1-6, wherein the taxane synthesis pathway, in particular taxol synthesis pathway, is derived from stem or trunk bark of the genus Taxus or Yew tree, in particular the Pacific yew, or Taxus brevifolia, Taxus baccata, Taxus cuspidurata, Taxus Canadensis, and Taxus floridana.

8. The method according to claims 1-6, wherein the gene or genes in the taxane synthesis pathway, in particular the taxol synthesis pathway, is(are) derived from a taxane-producing microorganism, in particular a taxol-producing fungus, in particular a strain of the genus Taxomyces, in particular Taxomyces andreanae, a strain of the genus Pestalotiopsis, in particular Pestalotiopsis microspora, or a strain of the genus Pestalotia, in particular Pestalotia heterocornis.

9. The method of claims 1-3, wherein the filamentous fungus host cell is of the genus Aspergillus, in particular a strain of Aspergillus niger, Aspergillus oryzae, or Aspergillus nidulans, Aspergilus japonicus, Aspergillus foetidus, Aspergillus aculeatus, or a strain of the genus Fusarium.

10. The method of claim 1-3, wherein the yeast host cell is derived from the genus Saccharomyces, in particular Saccharomyces cerevisiae.

11. The method of claim 1-10, wherein the host cell is taxane resistant, in particular taxol resistant and/or does not produce toxins.

12. An isolated DNA sequence comprising the taxane synthesis pathway, especially taxol synthesis pathway.

13. The expression vector comprising a DNA sequence of claim 12, wherein one or more genes in the pathway is(are) operably linked to one or more control sequences.

14. The vector of claim 13, wherein said DNA sequence is operably linked to a promoter sequence and optionally to a sequence encoding a secretion signal.

15. A host cell comprising a taxane synthesis pathway derived from a taxane-producing organism, plant or tree, wherein the taxane synthesis pathway is foreign to the host cell.

16. The host cell of claim 15, wherein the taxane synthesis pathway is operably linked to regulatory control elements, such as the native regulatory control elements of a taxane synthesis pathway.

17. The host cell of claims 15 or 16, wherein the host cell is of microbial, in particular fungal or bacterial origin, especially of yeast or filamentous fungus origin, or plant origin.

18. The host cell of claim 17, wherein the host cell is of the genus Aspergillus, in particular a strain of Aspergillus niger, Aspergillus oryzae, or Aspergillus nidulans, Aspergilus japonicus, Aspergillus foetidus, Aspergillus aculeatus, or the genus Fusarium.
